SEQUENCE LISTING (2 sequences)

SEQ ID NO: 1 - DNA, 17 bp, Artificial
  forward PCR Primer
gtaaaacgacggccagt

SEQ ID NO: 2 - DNA, 14 bp, Artificial
  reverse PCR Primer
acagctatgccatg

(19) United States
(12) Patent Application Publication
(10) Pub. No.: US 2005/0069893 A1
(43) Pub. Date: Mar. 31, 2005

(54) Diagnostic method for glaucoma

(76) Inventors: Josef Flammer, Binningen (CH); Olga Golubnitschaja, Bonn (DE)

Correspondence Address: JOYCE VON NATZMER, 4615 NORTH PARK AVENUE, SUITE 919, CHEVY CHASE, MD, 20815, US

(21) Appl. No.: 10/506,138

(22) PCT Filed: Mar. 1, 2002
(86) PCT No.: PCT/IB02/00648

(51) Int. Cl. C12Q 1/68

(52) U.S. Cl. 435/6

(57) ABSTRACT

The present invention relates to an ex vivo method for the diagnosis and/or prediction of glaucoma. Said method comprises detecting in a tissue and/or blood sample of a human individual an altered gene expression pattern of at least genes selected from the group of genes related to tissue remodeling. Furthermore, the invention relates to a DNA microarray comprising nucleic acid probes of genes of the specified gene group.

### TECHNICAL FIELD

The present invention relates to a method for the diagnosis and/or prediction of glaucoma as well as to an array of nucleic acid probes.

### BACKGROUND ART

Glaucoma is an optic neuropathy in which some retinal ganglion cells (RCG) die through an apoptotic process.

Primary Open Angle Glaucoma (“POAG”) is the most common form of glaucoma. The disease is characterized by the alteration of the trabecular meshwork, leading to obstruction of the normal ability of aqueous humor to leave the eye without closure of the space. A characteristic of such obstruction in this disease is an increased intraocular pressure (“IOP”), resulting in progressive visual loss and blindness if not treated appropriately and in a timely fashion.

Another form of glaucoma is characterized by progressive optic nerve damage and visual field loss with a statistically normal intraocular pressure (IOP≦21 mm Hg). This form of glaucoma is classified as normal tension glaucoma (NTG).

In the past, different diagnostic in vivo and ex vivo methods for the diagnosis of glaucoma has been described.

Patent application WO 98/44108 discloses in vivo and in vitro methods for diagnosing glaucoma wherein said methods are based on the determination of the expression of a trabecular meshwork induced glucocorticoid response protein (TIGR).

Patent application WO 98/36098 describes an in vitro method for the diagnosis of glaucoma based on the detection of a mutation in the gene cytochrome P450B1.

Although the above identified prior art describes diagnostic methods for glaucoma, there is currently no method available allowing an exact identification of patients with a predisposition for glaucoma development or for the progression of the disease.

There exists therefore an urgent need for a reliable method for the diagnosis and/or prediction of glaucoma and for means suitable for the use in said method.

### DISCLOSURE OF THE INVENTION

Hence, it is a general object of the invention to provide an ex vivo method for the diagnosis and/or prediction of glaucoma. Said method comprises detecting in a tissue and/or blood sample of a human individual an altered gene expression pattern of genes selected from at least the group of genes related to tissue remodeling.

The term “altered gene expression” encompasses an increased gene expression as well as a decreased gene expression of genes of interest compared to an average gene expression level observed in healthy subjects. The determination of the health state of a person is usually based on the subjective health state description of the patient, an interview by a physician and a physical examination of the patient.

The term glaucoma as used herein comprises all forms of glaucoma observed in the clinics.

In a preferred embodiment of the method said gene expression pattern further comprises genes selected from the following gene groups: genes related to DNA repair, genes related to cell adhesion, genes related to ischemia/reperfusion injury or genes in consequence of the glaucomatous damage.

In a further preferred embodiment of the method said gene expression pattern comprises a total of at least 4 genes, wherein at least one gene from each of the four gene groups. In a more preferred embodiment said gene expression pattern comprises a total of at least 8 genes, wherein at least 2 genes from each of the four gene groups.

It has to be understood that any combination of genes of said four gene groups is suitable for the use in the method according to the present invention. It is e.g. possible to use 2 genes of the first group, 1 gene of the second group, 4 genes of the third group and 3 genes of the fourth group.

Said altered gene expression of the genes of interest is preferably determined at the transcriptional level.

Preferred genes of the gene group which relate to tissue remodeling are the following genes:
metalloproteinases, metalloproteinase inhibitors and proteinase 3.

Preferred genes of the group of genes related to DNA-repair are the following genes:
XPGC, 14-3-3 σ (Stratifin), p53, NDR-X (ABC (ATP-binding cassette)-transporter), survivin, DEAD box X isoform protein (DBX), X-linked retinopathy protein, STM2 gene familial Alzheimer's disease, MRCK (myotonic dystrophy kinase-related cdc42 binding kinase), thioredoxin, NFkappB, inhibitor of apoptosis protein 1 (HIAP1, API1), IAP homolog C, TNFR2-TRAF signaling complex protein, MIHC, cyclin A1, guanine nucleotide-binding-protein G(I)/G(S)/G(T)beta subunit 1 (GNB1), transducin beta-1 subunit.

Preferred genes of the gene group which relate to cell adhesion are the following genes:
E-cadherin, cytochrome P450, cyclooxygenase-2, rho GDP dissociation inhibitor 1, rho GDI alpha, ARHGDIA, thymosin beta, VEGEFR 1, tyrosine protein kinase receptor SFLT, Phospholipase C gamma 1,1-phosphatidylinositol-4,5-bisphosphate-phosphodiesterase gamma 1, PLC-II, PLC-148, 68 kDa type I phosphatidylinositol-4-phosphate-5-kinase alpha kinase, 1-phosphatidylinositol-4-phosphate kinase, diphosphoinositide kinase, G protein-activated inward rectifier potassium channel 3, KIR 3.3, guanine nucleotide-binding protein G(I)/G(S)/G(T) beta-subunit 1, transducin beta-1 subunit, Rac alpha serine/threonine kinase, protein kinase B, c-akt, akt 1.

Preferred genes of the group of genes related to ischemia/reperfusion injury or genes in consequence of the glaucomatous damage are the following genes:
20S proteosome, NTP, Jun-D, c-jun N-terminal kinase (JNKK), JNK activating kinase 1 (JNKK1), MAP kinase 4 (MKK4), SRp20 splicing factor, lymphocyte-IgE-receptor, thromboxan A2 receptor, Na+/K+-ATPase, ITK, alkal. phosphatase.

In a particular preferred embodiment of the present invention said altered gene expression is determined in white blood cells, preferably peripheral lymphocytes, monocytes and stem cells.

Another object of the present invention is an array of nucleic acid probes immobilized on a solid support, wherein said array comprises nucleic acid probes of genes selected from the group of genes related to tissue remodeling.

Said array according to the present invention preferably further comprises nucleic acid probes of genes selected from the following gene groups: genes related to DNA repair, genes related to cell adhesion, genes related to ischemia/reperfusion injury or genes in consequence of the glaucomatous damage.

In a preferred embodiment said array comprises nucleic acid probes of genes selected from each of the above identified four gene groups.

Another preferred embodiment relates to an array which only comprises nucleic acid probes of genes selected from the four above defined gene groups.

An array according to the present invention can be used in an ex vivo method for the diagnosis and/or prediction of glaucoma, preferably in a method for the diagnosis and/or prediction of glaucoma according to the present invention.

A further object of the present invention is the use of genes of the above defined three gene groups for the somatic gene therapy of glaucoma.

### BRIEF DESCRIPTION OF THE DRAWINGS

The invention will be better understood and objects other than those set forth above will become apparent when consideration is given to the following detailed description thereof. Such description makes reference to the annexed drawings, wherein:

FIG. 1 shows the results of a Comet assay,

FIG. 2 shows the results of a dot blot assay (1: glaucoma patients, 2: healthy controls),

FIG. 3 shows the results of a RT-PCR amplification of XPGC transcripts,

FIG. 4a shows the results of a RT-PCR amplification of β-actin transcripts in control individuals,

FIG. 4b shows the results of a RT-PCR amplification of β-actin transcripts in glaucoma patients,

FIG. 4c shows the results of a RT-PCR amplification of maxtrix-metalloproteinase 9 (MMP-9) transcripts in control individuals,

FIG. 4d shows the results of a RT-PCR amplification of maxtrix-metalloproteinase 9 (MMP-9) transcripts in glaucoma patients,

FIG. 4e shows the results of a RT-PCR amplification of membrane type maxtrix-metalloproteinase 1 (MT1-MMP) transcripts in control individuals,

FIG. 4f shows the results of a RT-PCR amplification of membrane type maxtrix-metalloproteinase 1 (MT1-MMP) transcripts in glaucoma patients,

FIG. 4g shows the results of a RT-PCR amplification of metalloproteinase inhibitor 1 precursor 1 (TIMP-1) transcripts in control individuals,

FIG. 4h shows the results of a RT-PCR amplification of metalloproteinase inhibitor 1 precursor 1 (TIMP-1) transcripts in glaucoma patients,

FIG. 5a shows a restriction analysis of the 209 bp PCR fragment of β-actin,

FIG. 5b shows a restriction analysis of the 289 bp MMP-9 PCR fragment,

FIG. 5c shows a restriction analysis of the 295 bp MT1-MMP PCR fragment and

FIG. 5d shows a restriction analysis of the 393 bp TIMP-1 PCR fragment.

### MODES FOR CARRYING OUT THE INVENTION

The determination of expression patterns of specific genes according to the method of the present invention allows an exact diagnosis of patients with glaucoma as well as an exact identification of patients with a predisposition for chronic glaucoma development or for the progression of the disease. The method of the present invention offers a number of diagnostic advantages. For example, said method is highly sensitive and minimal-invasive by just taking/collecting a small tissue sample and/or a small amount of a body fluid, in particular blood, from a patient.

The man skilled in the art knows suitable gene expression detection methods which can be employed in a method of the present invention. Said methods comprise e.g. northern blot analysis, RT-PCR, real time quantitative PCR, immunohistochemical methods, ELISA, Dot blot analysis.

In a preferred embodiment the gene expression level is determined at the transcriptional level i.e. the amount of a RNA transcript is determined. A biological sample of a patient e.g. a tissue sample, preferably blood, is processed to isolate mRNA using one of the established methods for mRNA isolation and purification. The RNA is preferably isolated from peripheral blood leukocytes. The isolated mRNA is then transcribed to a DNA in a reaction with a reverse transcriptase. The resulting cDNA can then be analyzed by the method of the present invention. A particularly suitable method for the use in the present invention is RT-PCR which allows a fast determination of expression levels of genes. The primers for the RT-PCR are preferably chosen so that a non-conserved region of the genes are amplified.

A preferred means for the detection of said RNA transcripts is a DNA microarray. The construction of DNA microarrays and their use is well known in the art. For references see e.g. DNA Microarrays: A practical approach, Edited by M. Schena, Oxford University Press, Oxford, UK, 1999; Lemieux et al., Overview of DNA Chip Technology, Molecular Breeding 1998, 4, p. 277-289; and the internet site http://www.gene-chip.com and references cited therein.

An array comprises nucleic acid probes immobilized on a solid support. The term “nucleic acid probe” as used herein encompasses single stranded nucleic acids capable of binding to a target nucleic acid of complementary sequence by base pairing e.g. oligonucleotides, partial or complete cDNAs. A nucleic acid probe can include natural or modified bases. Nucleic acid probes can be between 10-500, 10-250, 10-150, 10-75, 10-50 and 10-25 bases long. The specific length of the used probes depends on the specific gene and said length has to be determined for each gene by the man skilled in the art.

In a preferred embodiment said nucleic acid probes stem from non-conserved domains of the protein of interest, more preferably from a non-conserved N-terminal domain or a non-conserved C-terminal domain of the protein of interest.

An exemplary embodiment of the method according to the present invention using a DNA array comprises the following steps: preparation of a sample of nucleic acids, hybridization of the sample of nucleic acids to an array, detection of hybridized nucleic acids and analysis of hybridization patterns.

Nucleic acid sample preparation typically includes the following steps: mRNA isolation and purification from a tissue and/or a body fluid sample, reverse transcription to cDNA and optionally second strand synthesis. Synthesized cDNA is typically labeled. Label can e.g. be introduced by one of the nucleotides being incorporated. Detectable labels suitable for use include e.g. spectroscopic, photochemical, biochemical or immunochemical means.

In one method of detection, denatured labeled nucleic acid derived from mRNA of the sample is applied to an array. Said nucleic acid hybridizes to complementary probes immobilized on the array and hybridization is identified by detecting label. The position of label is detected for each probe in the array and the concentration of each sequence that is complementary to a probe on the array is determined by measuring e.g. the fluorescence intensity using a reader. Comparison of the hybridization pattern of a patient sample to a control sample indicates which probes hybridize to nucleic acid strands that derive from mRNAs that are differentially expressed between the two samples. An expression pattern of the patient sample differing from the expression pattern of the control is indicative for glaucoma or a predisposition for glaucoma.

Genes of the above defined groups of genes related to glaucoma or anti-sense oligonucleotides thereof can be used for gene therapy of glaucoma.

For gene therapy a nucleic acid coding for a protein of the above identified groups is introduced into a suitable vector, preferably an adenoviral vector, allowing the expression of a said protein in the addressed target cells, preferably ganglion cells of the optical nerve. Such a vector suitable for gene therapy and allowing expression of the specific gene comprises the encoding nucleic acid under the control of a target cell specific promoter. Gene therapy methods and vector systems are e.g. described in Gene Therapy, T. Blankenstein, 1998 and Gene Therapy—from laboratory to the clinic, edited by Kam M. Hui.

The invention is now further illustrated by means of examples.

Experimental Part

Purpose

In order to investigate specific differences on the molecular level between patients with vasospastic syndrom, Normal Tension Glaucoma (NTG) patients and High Tension Glaucoma (HTG) patients have been compared to healthy controls.

Results

1. Comet Assay

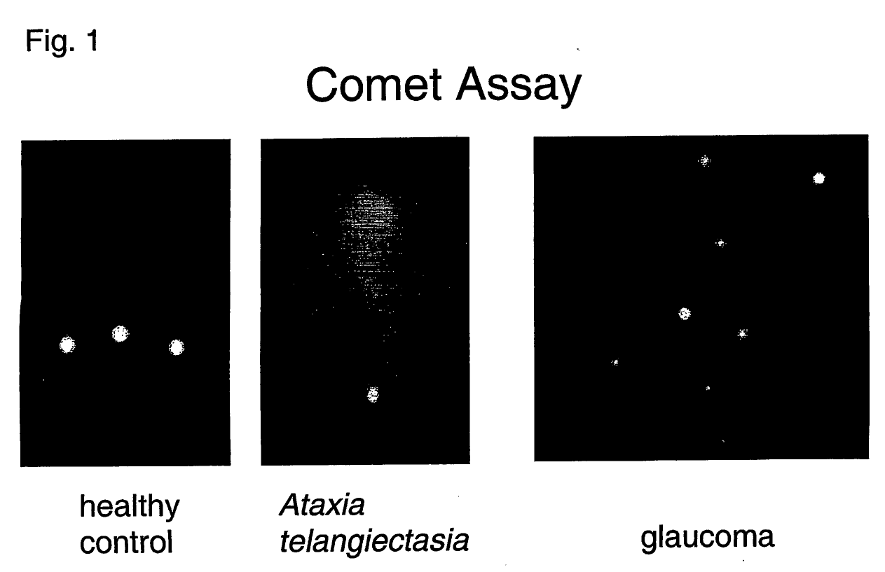

Evaluation of the initial DNA damage. Directly after thawing of the vital cells the majority (55%) of the healthy controls exhibited a slight degree in DNA damage (class 2), while only 5% of this group show severe DNA damage (class 4). No DNA damage (class 1) could be observed in 25%, while 15% of the healthy controls could be classified for class 3. In NTG patients a shift towards a higher degree of DNA damage could be observed. Cells derived from NTG patients could be mainly detected in the state of class 3 (intermediate DNA damage) with an percentage of 41%. Compared to controls, also the percentage of cells in the state with severe DNA damage (class 4) increased up to 26%. The amount of cells without DNA damage remained rather stable (29), while the amount of cells in the state class 2 decreased to 4%. The distribution pattern of HTG patients was rather similar to the group of NTG patients: the majority of the cells (47%) exhibited a DNA damage of the intermediate state (class 3), 21% of the cells exhibited no DNA damage, 10% a mild (class 2) and 22% a severe DNA damage (class 4). In FIG. 1 a distribution pattern of NTG-patients and healthy controls is shown. A patient with Ataxia telangiecasia served a positive control for DNA damage.

Evaluation of the DNA damage after in vitro-incubation. After thawing the cells got a regenerating period of 3 hours at 37° C. in phosphate buffered saline (PBS). Afterwards, the status of the DNA was examined. Compared to the evaluation directly after thawing the majority of the cells derived from healthy controls shifted towards undamaged DNA (31%; class 1) and mild DNA damage (26%; class 2), respectively. In addition, 27% of cells exhibited intermediate DNA damage (class 3) and 16% severe DNA damage (class 4). In contrary, within the group of NTG patients no increase of the amount of cells with undamaged DNA could be observed. In average there was following distribution: 13% with undamaged DNA (class 1), 29% with mild DNA damage (class 2), 32% with intermediate DNA damage (class 3) and 26% with severe DNA damage (class 4). Within the group of HTG patients the majority of the cells could exhibit the status of intermediate DNA damage (class 3) with 54% and severe DNA damage (class 4) with 36%. In average, 10% of the cells could be classified for class 1 (undamaged DNA) and 5% for class 2 (mild DNA damage).

2. Subtractive Hybridization and Dot Blots.

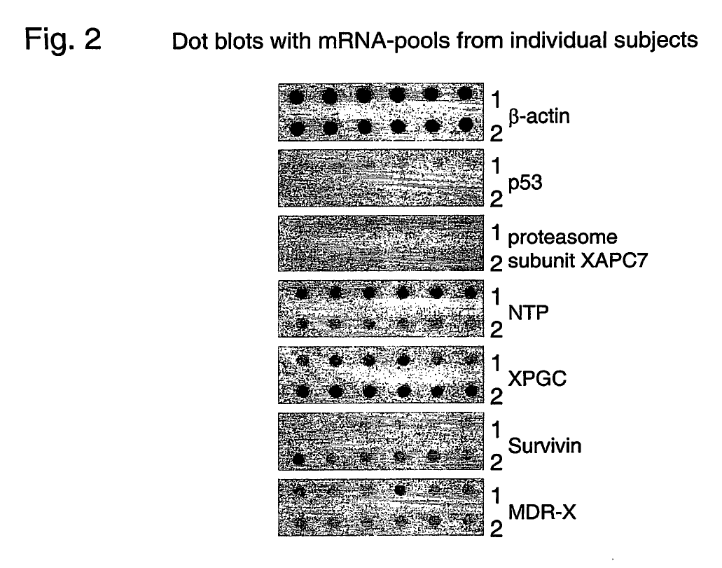

The subtracted cDNAs showed very similar pattern for all NTG patients. The subtracted cDNAs have been cloned and sequenced. The comparison of their sequences with data Genbank revealed homologies with genes coding for the following known proteins listed up in table 1 and 2. For better visualization of the different expression pattern of NTG patients compared to healthy controls dot blots were performed with 6 NTG patients and 6 healthy controls. Results are presented in FIG. 2: compared to controls NTG patients exhibited on the level of mRNA expression a slight increase in p53 as well as in 20S-proteasome subunit, and a stronger increase in the expression of the neuronal thread protein (NTP). In contrary, mRNA expression decreased for XPGC (Xeroderma Pig-mentosum group complementing factor), survivin and a new identified gene MDR-X.

TABLE 1
Homology of the up-regulated genes in lymphocytes
of NTG patients (hs-Homo sapiens)

| | EMBL | | Length of | % similarity |
|---|---|---|---|---|
| | accession | | the cDNA | of amino acid |
| Name of the gene | number | Organism | (bp) | sequence |
| p53 | X02469 | hs | 796 | 100 |
| cellular tumor | | | | |
| antigen | | | | |
| 20S-proteasome | AF022815 | hs | 168 | 99 |
| subunit | | | | |
| XAPC7 | | | | |
| neuronal thread | AF 010144 | hs | 213 | 87 |
| protein | | | | |
| AD7c-NTP (with | | | | |
| Alu-repeats- | | | | |
| containing | | | | |
| domains) related | | | | |
| to Alzheimer's | | | | |
| disease | | | | |

TABLE 2
Homology of the down-regulated genes in lymphocytes
of NTG patients (hs-Homo sapiens)

| | EMBL | | Length of | % similarity |
|---|---|---|---|---|
| | accession | | the cDNA | of amino acid |
| Name of the gene | number | Organism | (bp) | sequence |
| apoptosis | U75285 | hs | 312 | 100 |
| inhibitor | | | | |
| survivin gene | | | | |
| XPGC gene | X71347 | hs | 174 | 100 (cDNA) |
| hypothetical ABC | P44656 | Haemophilus | 327 | 83 |
| transporter ATP- | | influenza | | |
| binding protein | | | | |
| HI0354; MDR-X | | | | |

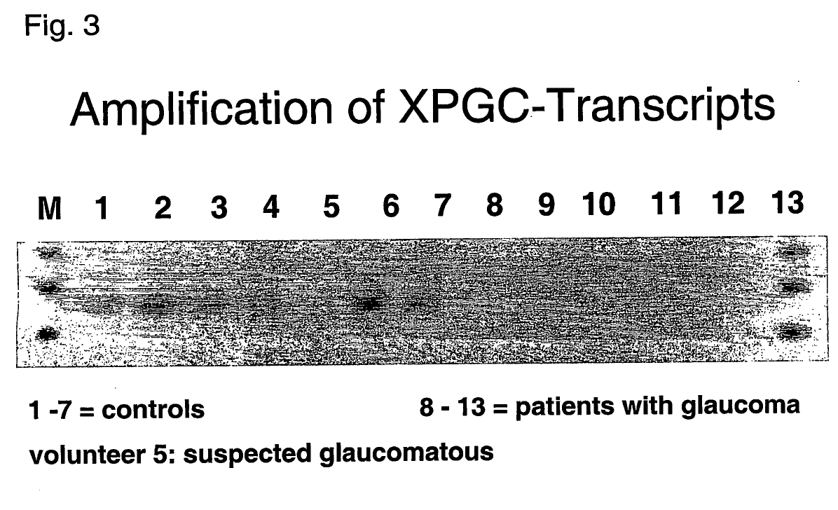

Amplification of the XPGC-Transcript. In addition to the dot blot experiments, RT-PCR was performed to evaluate XPGC gene expression. As shown in FIG. 3 all NTG patients exhibited a lack of XPGC gene expression. In contrary, in all healthy controls—with the exception of one volunteer (no. 5)—XPGC expression was detectable. Re-examination of volunteer no. 5 revealed a glaucomatous excavation of the optic nerve head, however the visual field was normal. This indicates that the person is a) at risk for developing glaucomatous damage or b) already suffers from preperimetry glaucoma.

3. Atlas cDNA Expression Arrays

The spectrum of screened genes of glaucoma patients vs. healthy controls was extended by using cDNA Expression Arrays in combination with an Imagine System. The results revealed an altered gene expression of 92 genes in glaucoma patients compared to controls. 33 of genes exhibited down-regulation, while 59 genes were upregulated. Focussing on metalloproteinases—a group of proteins, which are essential for tissue remodeling—following expression patterns of genes have been found to be altered in leukocytes of glaucoma patients: up-regulation of matrix-metalloproteinase 9 (MMP-9) and membrane-type matrix-metalloproteinase 1 (MT1-MMP), and dysregulation of metalloproteinase inhibitor 1 precursor 1 (TIMP-1). The results for MMP-9 and MT1-MMP could be confirmed by subtractive hybridization and real time QPCR (quantitative PCR) confirmed. Data of the screened genes mentioned above are listed up in table 3:

TABLE 3

| | | | | % similarity |
|---|---|---|---|---|
| | | | | of |
| | Genbank | | Length of | amino |
| | accession | | the cDNA | acid |
| Name of gene | number | Organism | (bp) | sequence |
| Matrix- | BC006093 | hs | 683 | 100 |
| metalloproteinase | | | | |
| 9 (MMP-9; | | | | |
| gelatinase B; | | | | |
| 92-kDa type IV | | | | |
| collagenase | | | | |
| precursor) | | | | |
| Membrane-type | X83535 | hs | 239 | 100 |
| MAtrix- | | | | |
| metalloproteinase | | | | |
| 1 (MT1-MMP, | | | | |
| MMP-14 precursor) | | | | |
| Metalloproteinase | X03124 | hs | done only | done only |
| inhibitor | | | by | by |
| 1 precursor 1 (TIMP- | | | expression | expression |
| 1) | | | array | array |

4. Confirmation of Target Gene Expression Using Specific RT-PCR

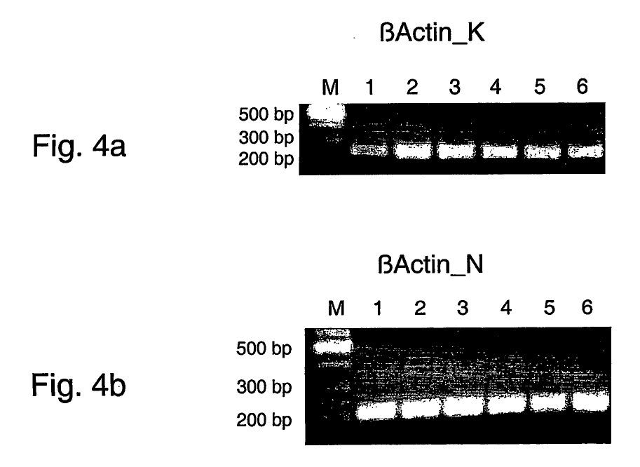

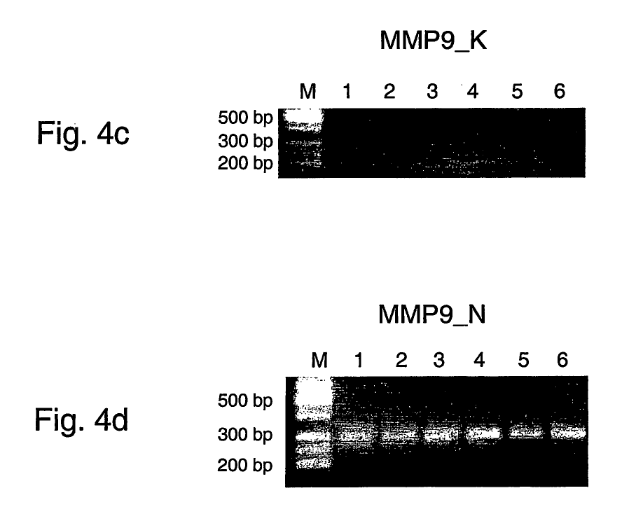

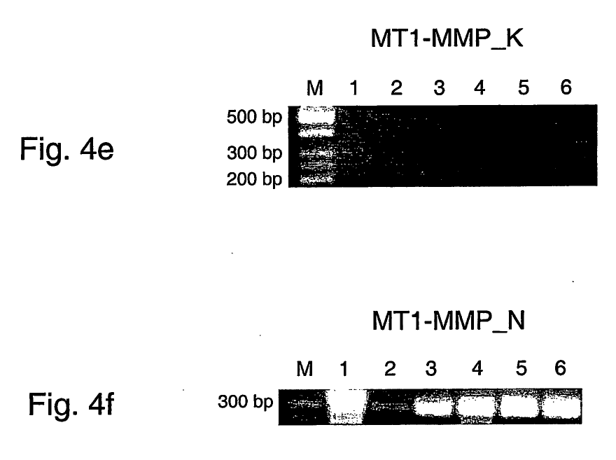

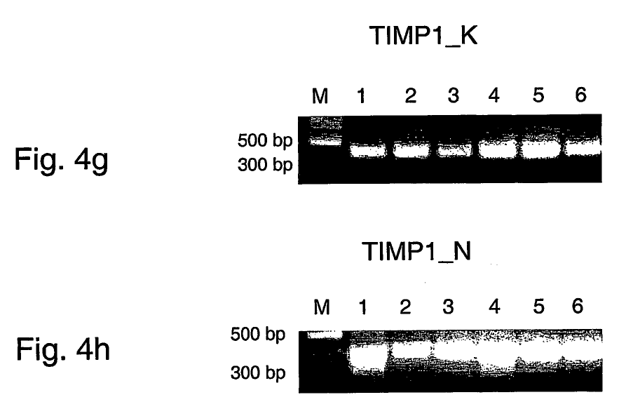

Amplification of cDNA fragments of MMP-9 and MT1-MMP by RT-PCR confirmed an induction of their expression in circulating leukocytes of glaucoma patients in contrast to healthy controls (FIG. 4C to F). As an internal control for cDNA synthesis the housekeeping gene β-actin was amplified (FIGS. 4A and B).

5. Restriction Analysis

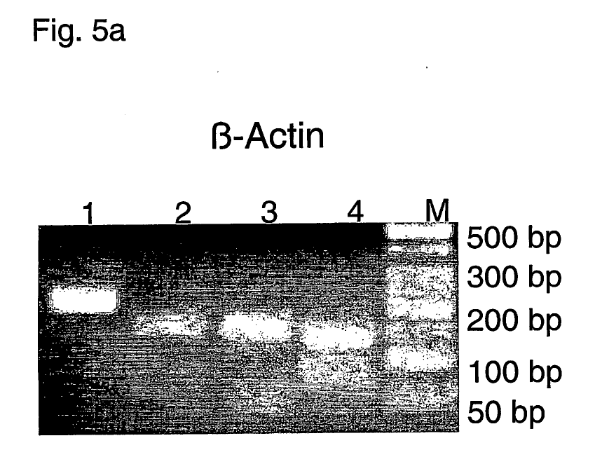

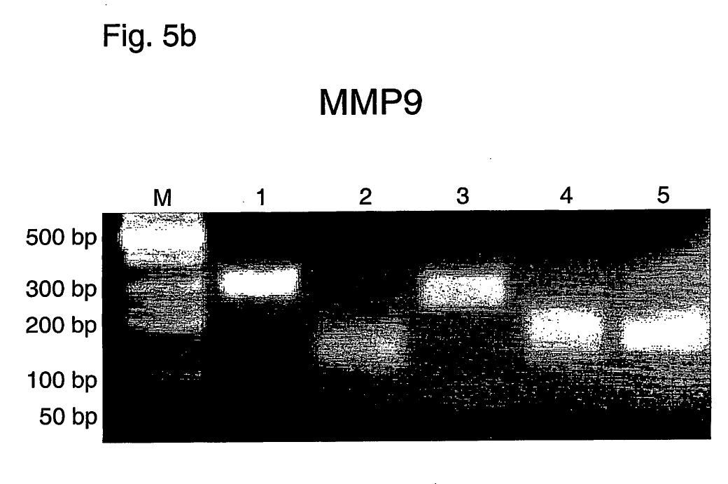

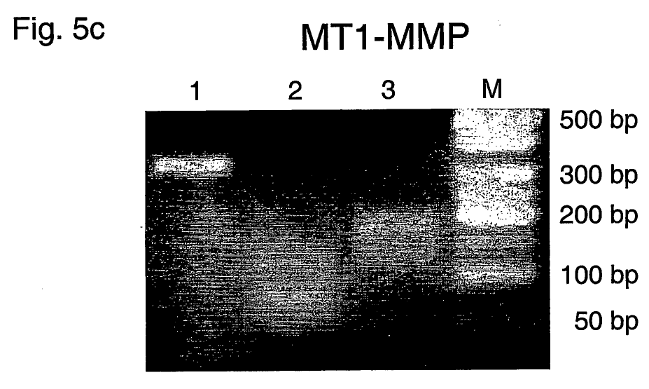

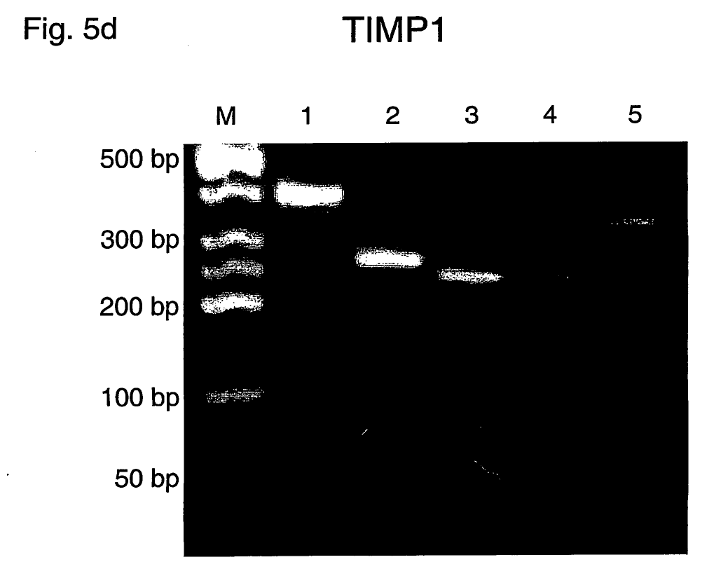

The amplification of the target PCR products has been confirmed by restriction analysis. Restriction analysis of the target RT-PCR products in 3% “wide range” agarose gel. In FIG. 5 lane 1 belongs to a non-digested amplification product; lane 2 and 3, and in addition 4 and 5 belong to amplifications products obtained by digestion with selected endonucleases. Restriction analysis was performed using AluI to get fragments with 58 and 151 basepairs ((bp); FIG. 5A), AvaI for fragments with 45 and 164 bp, HaeIII for fragments 15, 44 and 159 bp, RsaI for fragments 77 and 132 bp, all from the 209 bp beta-actin amplification product. To get fragments from the 289 bp MMP-9 amplification product restriction analysis was performed using AluI to get fragments with 40, 123 and 125 bp fragments, HpaII to get fragments with 46 and 243 bp, PvuII to get fragments with 123 and 166, and RsaI to get fragments with 142 and 147 bp (FIG. 5B). To get fragments from the 295 bp MT1-MMP amplification product restriction analysis was performed using HaeIII for fragments with 6, 54, 58, 69 and 108 bp, and RsaI for fragments with 125 and 170 bp (FIG. 5C). To get fragments from the 393 bp TIMP-1 amplification product restriction analysis was performed using HaeIII for fragments with 2, 30, 96 and 265 bp, HindIII for fragments with 154 and 239 bp, HpaII for fragments with 152 and 241 bp and PstI for fragments with 28, 29 and 336 (FIG. 5D).

5. Quantitative Analysis of Gene Expression Using Real-Time PCR

Relative gene expression was calculated basing on the individual C_{T} values of genes of interest and the housekeeping gene β-actin. Although in contrast to healthy volunteers, the leukocytes of all glaucoma patients demonstrated an expression of the MMP-9 gene, the transcription level differs up to 5 times among the extreme cases. Also the transcriptional level of TIMP-1 is very heterogeneous for these patients and differs from sample to sample up to 25×. Furthermore there is no correlation in increase of transcription between MMP-9, and TIMP-1. MT1-MMP is highly expressed in 5 glaucoma patients and weak expressed in one patient.

### Conclusion

1. Depending on the degree of fragmented DNA normal tension glaucoma (NTG) patients show a less sufficient ability of DNA repair compared to normals.

2. These patients also differ from normals in the expression pattern of various genes. The genes belongs to the gene families involved in a) tissue remodeling, b) DNA repair, c) ischemia-reperfusion and d) adhesion.

Materials and Methods

1. Blood Samples

Blood samples were collected from patients with NTG and HTG as well as from healthy controls. All glaucoma patients had bilateral typical glaucomatous optic nerve head cupping and visual field defects. In NTG patients intraocular pressure (IOP) never exceeded 21 mm Hg, but after local cooling of the fingers all these NTG patients exhibited a stop in blood flow for more than 20 sec, which was detected by nailfold capillaromictroscopy (indicative for vasospasm). In contrast, HTG patients exhibited an IOP higher than 21 mm Hg, but no vasospastic response. Ophthalmological examination of healthy controls yielded unremarkable results and also no vasospastic response. No patient had received either a systemic or a locally applied ocular therapy at least four weeks before blood draw.

2. Leukocyte Isolation

Leukocytes were isolated from heparinized blood by density gradient centrifugation as previously described (Kalmar et al., 1988). After isolation pellets of PBS-washed leukocytes were stored at −70° C. either as dry pellet or frozen in DMSO-containing culture medium as vital cells.

3. Comet Assay

Sample preparation. The rate of cells containing fragmented DNA was evaluated by the use of a Comet assay. The principle of Comet assay (Trevigen INC., USA) or single cell electrophoresis is based on the ability of denatured, cleaved DNA fragments to migrate our of the cells under the influence of an electric field. Undamaged DNA migrates slower and remains within the confines of the nucleus when current is applied. Evaluation of the DNA “comet” tail shape and migration pattern allows for assessment of DNA damage (FIG. 1). In detail the method was described by Ostling & Johanson (1984). In brief, isolated leukocytes in a density of 200-300 cells per sample were immobilized in a bed of low melting agarose. After cell lysis samples were treated with alkali to unwind and denature the DNA and hydrolyze sites of damage. After electrophoresis samples were stained with SYBR Green, a fluorescent DNA intercalating dye.

Sample analysis. The comets were visualized under the fluorescent microscope (Olympus) at a magnification of 200×. Excitation wavelength of 515-560 nm and a barrier filter for 590 nm were used. At least 100 comets were analyzed for each data point. For quantification of the DNA damage the total length of the comet (head and tail) were measured and the degree of the damage was calculated according to the criteria of McKelvey-Martin et al. (1993). The degree of the damage was assigned to 4 classes (1-4) based on the visual aspect of the comets, considering the extent of DNA migration (Visvardis et al., 1997). As shown in FIG. 1 comets with a bright head and no tail were classified as class 1 (intact DNA) while comets with a small head and a long diffuse tail were classified as class 4 (severely DNA damage). Intermediate characteristics were assigned to class 2 and 3. Cell loss greater then the average calculated for healthy donors was assigned to class 5. Initial DNA damage (DD) and DNA damage after incubation in BPS for 3 hours at 37° C. (DD₃) were estimated quantitatively using the modified equation (1) described by Jaloszynski et al. (1997):
DD=(n₂+2n₃+3n₄+4n₅)/(S/100),
where DD: DNA damage, n₂-n₄: amount of calculated comets in class 2, 3 and 4, respectively; S: total number of scored comets including class 1.

Statistical analysis. Initial DNA damage and repair capacity after 3 hours of incubation were compared between three groups by nonparametric two-way ANOVA. All statistical analysis were done using the Graphpad Prism software (version 2.01). Statistical significance was calculated by the two sided, unpaired Student's t-test.

4. “Gene Hunting” by Subtractive Hybridization

Isolation of mRNA. Isolation of mRNA was performed using the Quick Prep Micro mRNA Purification Kit (Pharmacia Biotech, Uppsala, Sweden) according to the manufactures protocol. Quality-check was performed by First-strand cDNA Synthesis Kit (Pharmacia Biotech, Uppsala, Sweden) using the incorporation of [α-³²P] dATP (Amersham, Buckinghamshire, UK) with subsequent electrophoresis on a 1% agarose gel followed by autoradiography (Sambrock et al., 1981). The reflection film (NEN Life Science Products) was exposed to the gel four 2 hours at room temperature.

Construction of the subtractive library. In principle, construction of the subtractive library is based on the cloning of the transcripts (in form of cDNA) of those genes, which are activated/suppressed to become up-or down-regulated under pathological conditions. To identify these genes two pools of transcripts are used: the complete pool of mRNA from patients and the complete pool of mRNA from healthy controls. Both pools of transcripts—called induced and uninduced pool of mRNA—undergo the molecular biological comparison with the subsequent subtraction of the difference between two pools representing the transcripts activated or suppressed due to the disease. Construction of the subtractive library was done as follows: mRNA from leukocyte samples and controls were biotinylated by UV radiation according to the instruction manual of the Subtractor Kit (Invitrogen, Leek, N L). To avoid false positive results the mRNA of the uninduced pool was added in excess. Equal quantity of each mRNA pool was subjected to reverse transcription with subsequent denaturation of mRNA-template. Each newly synthesized cDNA pool (induced pool) was hybridized with the corresponding uninduced biotinylated mRNA-pool at 68° C. for 48 hours. The hybridization mixture was incubated with streptavidin and thus all the biotinylated molecules (uninduced as well as RNA/DNA hybrids) were complexed with streptavidin. The streptavidin nucleic acid-complexes were removed by phenol-chloroform extraction and subtracted cDNAs were precipitated with ethanol (Sive & John, 1988). For each pair of NTG patient/control both pools were subtracted: the induced “NTG-genes” and the induced “normal genes”. The 2^{nd} strand cDNA synthesis was performed with the cDNA Synthesis Kit (Boehringer Mannheim, FRG). The aim of the constructed libraries was to compare gene expression individually and in the groups of NTG-patients with healthy controls. Only those genes which have been subtracted from both the individual pairs and the corresponding groups have been considered as relevant.

Cloning of subtracted cDNA. Subtracted cDNAs were cloned by using the pSPORT 1 cloning vector (GIBCO, Life Technologies, Eggenstein, FRG) according to the cloning methods described by Sambrook et al. (1989). To enable visualization of the subtracted cDNAs the cloned cDNAs were amplified using the universal primers I-5′ GTAAAACGACGGCCAGT 3′ (Seq. Id. No. 1) and II-5′ ACAGCTATGACCATG 3′ (Seq. Id. No. 2) restricting the multiple cloning site of pSPORT 1 vector. The amplificates were analyzed in a 1% agarose gel. The corresponding cDNAs were cut off from the gel cleaned with the DNA Clean Kit (AGS, Heidelberg, FRG) and recloned in Sma I-site of pUC 18 vector. The recombinant molecules were used for transformation in INVaF Eschericia coli cells (Invitrogen, Leek, NL). Recombinant plasmid DNAs were analysed for the length of the inserted fragment using restriction analysis. Plasmid DNAs were purified using QiaFilter Plasmid Midi System (Quiagen, Hilden, FRG). Plasmid DNAs were sequenced by MWG-Biotech (Ebersberg, FRG).

Gene identification. Homologies were determined by computer assisted comparison of data with DNA and protein gene banks (EMBL and SWISS-PROT, Heidelberg, FGR). Alignments were prepared using “DNASIS”-programs from MWG-Biotech (Ebersberg, FRG).

Dot blot analysis. For the quantification of the specific transcripts the individual cloned and sequenced cDNAs have been used as specific labeled probes for dot blot hybridization. After cloning and purification each probe was denatured prior labelling at 95° C. for 5 min and subsequently labeled with fluorescein-12-dUTP using the Renaissance Random Primer Fluorescein-12-UTP Labeling Kit (NEN Life Science Products). Aliquots of the isolated mRNA-pools have been applied for the hybridization with the specific probes using dot blots technique according to the protocol of White & Bancroft (1982). In brief, samples of the mRNA-pools were placed onto a positively charged nylon membrane. After fixation, the nylon membrane was incubated in a pre-hybridization solution containing a block reagent (NEN Life Science Products) for 3 hours at 65° C. in a hybridization oven. The membrane was hybridized step-wise with each labeled probe overnight at 65° C. After hybridization, non-specifically bound material was removed by washing the membrane two times with pre-hybridization butter. The membrane was then blocked with blocking reagent and incubated with antifluorescein HRP-antibody (1:1000; (NEN Life Science Products) for 1 hour at 37° C. After washing the membrane was incubated in Nucleic Acid Chemiluminiscence Reagent (NEN Life Science Products) for 1 hour and afterwards exposed to an autoradiography reflection film (NEN Life Science Products) for 1 hour at room temperature. Each hybridization was performed in the same manner. Between the individual hybridisations the membrane was stripped according to the manufacturer's protocol. Densiometry of the films was performed using a densiometer and the quantification software program from MWG-Biotech (FRG).

Statistical analysis. The ANVA with subsequent Kruskal Wallis Test and Student's t-test were applied and linear regression analysis was performed. The level of significance was at p<0.05.

5. “Gene Hunting” by Hybridization of cDNA Probes to Expression Arrays

Atlas™ Human 1.2 Array (Clontech, Palo Alto, USA) designed for the evaluation of different molecular expression patterns was used. The Atlas™ Human 1.2 Array includes 1176 human cDNAs, nine housekeeping control cDNAs, and negative controls all immobilized on a nylon membrane. Synthesis of cDNA from isolated mRNA derived from glaucoma patients and healthy controls was performed using the First-Strand cDNA Synthesis Kit from Pharmacia (Uppsala, S). After synthesis and labeling with fluorescein-12-dUTP (see above) the Atlas™ Human 1.2 Arrays was hybridized with each individual labeled cDNA probe. After hybridization non-specific bound material was removed by several washing steps and the membrane was then blocked with blocking reagent. Afterwards, the membrane was incubated with anti-fluorescein HRP-antibody (NEN Life Science Products) for 1 hour at 37° C. followed by a washing procedure and the incubation with the chemiluminiscence reagent (NEN Life Science Products). Then, the membrane was exposed to an autoradiography reflection film (NEN Life Science Products) for 1 hour at room temperature. Evaluation was performed using the Atlas Image 2.0 software developed specifically for the analysis of Atlas cDNA Expression Arrays (Clontech, Palo Alto, USA).

6. Polymerase Chain Reactions

Reverse Transcriptase Polymerase Chain reaction (RT-PCT). In order to detect qualitatively an expression of the target genes and to optimize individual reaction conditions for the Real-Time Quantitative PCR (see below) RT-PCR was performed with specific primers designed for MMP-2, MMP-9, MT1-MMP and TIMP1 genes. Synthesis of cDNA from isolated mRNA derived from glaucoma patients and healthy controls was performed using the First-Strand cDNA Synthesis Kit from Pharmacia (Uppsala, S). PCRs were performed using a hot-start Taq-polymerase (Abgene, Hamburg, FRG) and were run for 35 cycles. PCRs without DNA served as negative controls and PCRs with sequences templates as positive controls. For analysis ethidium bromide-stained PCR products, which had been separated in an agarose gel, were visualized under UV illumination.

Real-Time Quantitative PCR (RT-QPCR). In order to profile changes in the expression of genes of interest RT-QPCR has been performed by using SYBER Green I as intercalation dye and fluorescent reporter molecule to detect the accumulation of amplified double-stranded products in an iCycler iQ™ Detection System (Bio-Rad LAboratories, USA). RT-PCR was performed as described above, only with one exception: hot-red Taq-polymerase (Abgene, Hamburg, FRG) was substituted by Taq DNA polymerase (Roche, CH) to avoid color signal disturbances. The algorithm of the iCycler iQ™ Detection System normalizes the reporter signal (non-intercalated SYBER Green) to a passive reference and multiplies the SD of the background signal in the first few cycles by a defaulter factor of 10 to determine the threshold. The cycle at which the baseline level is exceeded is defined as threshold cycle (CT). CT depends on the initial template copy number and is proportional to the log of the starting amount of nucleic acid (Heid et al.). By subtracting differences of the CT values between the genes of interest and the housekeeping genes (β-actin) the data have been normalized. Relative levels were calculated for gene expression in NTG samples to control samples based on the differences in CT values (Heid et al., 1996).

Statistical analysis. All values are expressed as mean ±SEM. Values were compared using Student's t-test for parametric data. A p value of less than 0.05 was considered as significant.

Restriction analysis. Target PCR products were identified using specific restriction analysis. The target amplification products underwent an extraction from the agarose gel using the DNA isolation kit (DNA-Clean™, Hybaid-AGS, FRG) before digestion. They were digested in a final volume of 50 μl with 20 units of each restriction endonuclease for 4 hours, according to the protocol of the manufacturer (Roche, CH). Digested DNA fragments were separated in an agarose gel and visualized after staining with ethidium bromide by UV-light (FIG. 5).

While there are shown and described presently preferred embodiments of the invention, it is to be distinctly understood that the invention is not limited thereto but may be otherwise variously embodied and practiced within the scope of the following claims.

### REFERENCES

Heid C A, Stevens J, Livak K J, Williams P M. Real time quantitative PCR. Genome Res 1996 October; 6(10): 986-94
Jaloszynski P, Kujawski M, Czub-Swierczek M, Markowska J, Szyfter K, Bleomycin-induced DNA damage and its removal in lymphocytes of breast cancer patients studied by comet assay. Mutat Res 1997 December; 385(3): 223-33
Kalmar J R, Arnold R R, Warbington M L, Gardner M K. Superior leukocyte separation with a discontinuous one-step Ficoll-Hypaque gradient for the isolation of human neutrophils. J Immunol Methods 1988 Jun. 13;110(2): 275-81
McKelvey-Martin V J, Green M H, Schmezer P, Pool-Zobel B L, De Meo M P, Collins A. The single cell gel electrophoresis assay (comet assay): a European review. Mutat Res 1993 July; 288/1):47-63
Ostling 0, Johanson K J. Microelectrophoretic study of rediation-induced DNA damages in individual mammalian cells. Biochem Biophys Res Commun 1984 Aug. 30; 123(1): 291-8
Sambrok J, Fritsch E F, Maniatis T. Molecular cloning: A laboratory manual. In: Cold Spring Harbor Laboratory, Cold Spring Harbor. 1989; second edition: 1.53-1.73
Sive H L & St John T. A simple subtractive hybridization technique employing photoactivatable biotin and phenol extraction. Nucleic Acids Res 1988 Nov. 25; 15(22): 10937
White B A & Bancroft F C. Cytoplasmic dot hybridization. Simple analysis of relative mRNA levels in multiple small cell or tissue samples. J Biol Chem 1982 Aug. 10; 257(15): 8569-72
Visvardis E E, Tassiou A M, Piperakis S M. Study of DNA damage induction and repair capacity of fresh and cryopreserved lymphocytes exposed to H₂O₂ and gamma-irradiation with the alkaline comet assay. Mutat Res 1997 Jan. 31; 383(1): 71-80.

1. An ex vivo method for the diagnosis and/or prediction of glaucoma comprising
detecting in a tissue and/or blood sample of a human individual an altered gene expression pattern of genes selected from at least a group of genes related to tissue remodeling.

2. The method according to claim 1, wherein said gene expression pattern further comprises genes selected from a group of genes related to DNA repair and/or a group of genes related to cell adhesion and/or a group of genes related to ischemia/reperfusion injury or genes in consequence of the glaucomatous damage.

3. The method according to claim 2, wherein said gene expression pattern comprises a total of at least 4 genes, at least one from each of the four gene groups.

4. The method according to claim 1, wherein said altered gene expression pattern is determined at a transcriptional level.

5. The method according to claim 1, wherein the group of genes related to tissue remodeling comprises the following genes:
metalloproteinases, metalloproteinase inhibitors and proteinase 3.

6. The method according to claim 2, wherein the group of genes related to DNA-repair comprises the following genes:
XPGC, 14-3-3 σ, p53, MDR-X, survivin, DEAD box X isoform protein (DBX), X-linked retinopathy protein, STM2 gene familial Alzheimer's disease, MRCK (myotonic dystrophy kinase-related cdc42 binding kinase), thioredoxin, NFkappB, inhibitor of apoptosis protein 1 (HIAP1, API1), IAP homolog C, TNFR2-TRAF signaling complex protein, MIHC, cyclin A1, guanine nucleotide-binding-protein G(I)/G(S)/G(T)beta subunit 1 (GNB1), transducin beta-1 subunit.

7. The method according to claim 2, wherein the group of genes related to cell adhesion comprises the following genes:
E-cadherin, cytochrome P450, cyclooxygenase-2, rho GDP dissociation inhibitor 1, rho GDI alpha, ARHGDIA, thymosin beta, VEGEFR 1, tyrosine protein kinase receptor SFLT, phospholipase C gamma 1,1-phosphatidyl-inositol-4,5-bisphosphate-phosphodiesterase gamma 1, PLC-II, PLC-148, 68 kDa type I phosphatidyl-inositol-4-phosphate-5-kinase alpha kinase, 1-phosphatidylinositol-4-phosphate kinase, diphospho-inositide kinase, G protein-activated inward rectifier potassium channel 3, KIR 3.3, guanine nucleotide-binding protein G(I)/G(S)/G(T) beta-subunit 1, transducin beta-1 subunit, Rac alpha serine/threonine kinase, protein kinase B, c-akt, akt 1.

8. The method according to claim 2, wherein the group of genes related to ischemia/reperfusion injury or in consequence of glaucomatous damage comprises the following genes:
20S proteosome, NTP, Jun-D, c-jun N-terminal kinase (JNKK), JNK activating kinase 1 (JNKK1), MAP kinase 4 (MKK4), SRp20 splicing factor, lymphocyte-IgE-receptor, thromboxan A2 receptor, Na+/K+-ATPase, ITK, alkaline phosphatase.

9. The method according to claim 1, wherein said altered gene expression Pattern is determined in white blood cells.

10. An array of nucleic acid probes immobilized on a solid support, wherein said array comprises at least nucleic acid probes of genes selected from a group of genes related to tissue remodeling.

11. The array according to claim 10, wherein said array further comprises nucleic acid probes of genes selected from a group of genes related to DNA repair and/or a group of genes related cell adhesion and/or a group of genes related to ischemia/reperfusion injury or genes in consequence of the glaucomatous damage.

12. The array according to claim 11, wherein the nucleic acid probes are selected from genes for
metalloproteinases, metalloproteinase inhibitors and proteinase 3,
XPGC, 14-3-3 σ, p53, MDR-X, survivin, DEAD box X isoform protein (DBX), X-linked retinopathy protein, STM2 gene familial Alzheimer's disease, MRCK (myotonic dystrophy kinase-related cdc42 binding kinase), thioredoxin, NFkappB, inhibitor of apoptosis protein 1 (HIAP1, API1), IAP homolog C, TNFR2-TRAF signaling complex protein, MIHC, cyclin A1, guanine nucleotide-binding-protein G(I)/G(S)/G(T)beta subunit 1 (GNB1), transducin beta-1 subunit,
E-cadherin, cytochrome P450, cyclooxygenase-2, rho GDP dissociation inhibitor 1, rho GDI alpha, ARHGDIA, thymosin beta, VEGEFR 1, tyrosine protein kinase receptor SFLT, phospholipase C gamma 1,1-phosphatidyl-inositol-4,5-bisphosphate-phosphodiesterase gamma 1, PLC-II, PLC-148, 68 kDa type I phosphatidyl-inositol-4-phosphate-5-kinase alpha kinase, 1-phosphatidylinositol-4-phosphate kinase, diphospho-inositide kinase, G protein-activated inward rectifier potassium channel 3, KIR 3.3, guanine nucleotide-binding protein G(I)/G(S)/G(T) beta-subunit 1, transducin beta-1 subunit, Rac alpha serine/threonine kinase, protein kinase B, c-akt, akt 1, and
20S proteosome, NTP, Jun-D, c-jun N-terminal kinase (JNKK), JNK activating kinase 1 (JNKK1), MAP kinase 4 (MKK4), SRp20 splicing factor, lymphocyte-IgE-receptor, thromboxan A2 receptor, Na+/K+-ATPase, ITK or alkaline phosphatase.

13. The array according claim 11, wherein said array consists essentially of nucleic acid probes of genes of said gene groups.

14. (Cancelled)

15. (Cancelled)

16. (Cancelled)

17. The method of claim 3, wherein said gene expression pattern comprises a total of at least 8 genes.

18. The method of claim 6, wherein said white blood cells are peripheral lymphocytes, monocytes or stem cells.

19. An ex vivo method for the diagnosis and/or prediction of glaucoma comprising:
providing the array of claim 10, and
detecting in a tissue and/or blood sample of a human individual an altered gene expression pattern.

20. An ex vivo method for the diagnosis and/or prediction of glaucoma comprising:
providing the array of claim 11, and
detecting in a tissue and/or blood sample of a human individual an altered gene expression pattern.

21. An ex vivo method for the diagnosis and/or prediction of glaucoma comprising:
providing the array of claim 12, and
detecting in a tissue and/or blood sample of a human individual an altered gene expression pattern.

22. A method for treating a patient diagnosed with glaucoma comprising:
administering to said patient an effective amount of at least one gene of the following groups of genes:
(1) genes related to tissue remodeling,
(2) genes related to DNA repair,
(3) genes related to cell adhesion,
(4) genes related to ischemia/reperfusion injury or genes expressed in consequence of the glaucomatous damage, or
combinations thereof,
wherein said method is somatic gene therapy.

23. The method of claim 22, wherein
the group of genes in (1) comprises metalloproteinases, metalloproteinase inhibitors and proteinase 3,
the group of genes in (2) comprises XPGC, 14-3-3 σ, p53, MDR-X, survivin, DEAD box X isoform protein (DBX), X-linked retinopathy protein, STM2 gene familial Alzheimer's disease, MRCK (myotonic dystrophy kinase-related cdc42 binding kinase), thioredoxin, NFkappB, inhibitor of apoptosis protein 1 (HIAP1, API1), IAP homolog C, TNFR2-TRAF signaling complex protein, MIHC, cyclin A1, guanine nucleotide-binding-protein G(I)/G(S)/G(T)beta subunit 1 (GNB1) and transducin beta-1 subunit,
the group of genes in (3) comprises E-cadherin, cytochrome P450, cyclooxygenase-2, rho GDP dissociation inhibitor 1, rho GDI alpha, ARHGDIA, thymosin beta, VEGEFR 1, tyrosine protein kinase receptor SFLT, phospholipase C gamma 1,1-phosphatidyl-inositol-4,5-bisphosphate-phosphodiesterase gamma 1, PLC-II, PLC-148, 68 kDa type I phosphatidyl-inositol-4-phosphate-5-kinase alpha kinase, 1-phosphatidylinositol-4-phosphate kinase, diphospho-inositide kinase, G protein-activated inward rectifier potassium channel 3, KIR 3.3, guanine nucleotide-binding protein G(I)/G(S)/G(T) beta-subunit 1, transducin beta-1 subunit, Rac alpha serine/threonine kinase, protein kinase B and c-akt, akt 1; and
the group of genes in (4) comprises 20S proteosome, NTP, Jun-D, c-jun N-terminal kinase (JNKK), JNK activating kinase 1 (JNKK1), MAP kinase 4 (MKK4), SRp20 splicing factor, lymphocyte-IgE-receptor, thromboxan A2 receptor, Na+/K+-ATPase, ITK or alkaline phosphatase.
